(19) United States
(12) Patent Application Publication
(10) Pub. No.: US 2010/0280583 A1
(43) Pub. Date: Nov. 4, 2010

(54) Electrode Element, Electrode Lead Comprising An Electrode Element, Method For The Production Of An Electrode Lead

(75) Inventors: Klaus Bartels, Berlin (DE); Thomas Guenther, Michendorf OT Wilhelmshorst (DE); Tassilo Landgraf, Berlin (DE); Carsten Fruendt, Berlin (DE)

Correspondence Address: BUCHANAN INGERSOLL & ROONEY PC, P.O. BOX 1404, ALEXANDRIA, VA, 22313-1404, US

(73) Assignee: Biotronik CRM Patent AG, Baar (CH)

(21) Appl. No.: 12/766,097

(22) Filed: Apr. 23, 2010

(30) Foreign Application Priority Data
Apr. 29, 2009 (DE) 10 2009 002 707.6

(51) Int. Cl. A61N 1/05; A61N 1/04; H01R 43/20

(52) U.S. Cl. 607/116; 607/115; 29/876

(57) ABSTRACT

An electrically active electrode element for an implantable electrode lead, having an electrode, which includes an electrically active electrode surface facing toward the outside, and an elongated electric feed line, which is capable of establishing an electric connection to an electrically active implant at the proximal end thereof, and which is embodied as an electrically conducting cable end-to-end, wherein the cable forms the electrode at the distal end thereof. A related electrode lead having such an electrode element is provided, and a method for the production of said electrode line utilizing the electrode element is also provided.

### CROSS-REFERENCE TO RELATED APPLICATIONS

This application claims the priority of German Patent Application No. DE 10 2009 002 707.6, filed on Apr. 29, 2009 in the German Patent Office, the disclosure of which is incorporated herein in its entirety by reference.

### FIELD OF THE INVENTION

The present invention relates both to an electrically active electrode element for an implantable electrode lead comprising an electrode having an electrically active electrode surface facing toward the outside, and to an elongated electric feed line, which may establish an electrical connection at the proximal end thereof to an electrically active implant, and which is embodied as an electrically conductive cable end-to-end.

### BACKGROUND OF THE INVENTION

In the broadest sense, electrode elements are already known from the prior art. U.S. Pat. No. 7,174,220 already discloses an electrode element as a combination of an electrode and an elongated feed line, which electrically connects the electrode to an electrode plug at the proximal end of an electrode lead. For this purpose, the feed line is not the helical feed line commonly known to the person skilled in the art, but an elongated longitudinal feed line, which establishes a connection between the electrode and the plug in a direction that is as elongated as possible. This improves the flexibility and bendability that an implantable electrode lead requires, for which such an electrode element is suitable. Furthermore, said elongated construction reduces the diameter of an electrode lead.

It is known from the cited patent to produce the lead from a cable. A cable, in the sense of the present invention, is an elongated, flexible and elastic element consisting of individual wires that are twisted together, in this case for transmitting energy. The cable is produced from one or more strands (typically 1, 3, 7, or 12 strands) by means of “braiding,” which are created from individual wires (typically 3, 7, 19 individual wires) in a separated manufacturing step by means of “stranding.” For this purpose the individual wires may consist of solid matter or of so-called casing wire or DFT wires (DFT=drawn filled tube), wherein the latter wires are wires having a core material and a casing material differing from the core material. Material that is suitable for the solid wire and the core material preferably includes, but is not limited to, biocompatible and bio-stable metals, such as platinum iridium alloys, tantalum, niobium, zirconium, and molybdenum, and including the alloys thereof. Further suitable in casing wires, and generally particularly for the casing material, are platinum iridium alloys, and cobalt chromium base alloys for the core material, such as MP35N.

The ratios of the cross-sectional surface of core to casing with regard to the casing wire are typically 25-95%.

An electrode, in the sense of the present invention, is an electrically active element suitable for emitting electrical energy or electric impulses (such as stimulation impulses or defibrillation shocks), or for receiving signals from the environment of the electrode and transmit the same. According to the prior art, such as in U.S. Pat. No. 7,174,220, such stimulation and cognition electrodes are made from a rigid metal sleeve (such as pipe sections), which is electrically connected to the feed line by means of welding (laser welding, resistance welding, and the like), soldering, or crimping. In another embodiment according to the prior art, an electrode, preferably a shock electrode, consists of an exposed helical section of a helical feed line. Such a configuration results in a large diameter and little flexibility.

In order for an electrode to be able to emit energy or impulses, or receive body signals, such as temperature, impedance, heart or brain signals, etc., the electrode has an electrically active surface facing toward the outside—that is to say in the direction of the electrode environment, which is not insulated from the environment in the installation state into an electrode lead.

However, the described electrode element from the prior art has the particular disadvantage that the feed line, or the transition between the feed line and the electrode, has an increased tendency to break. High and perpetual bending loads act upon the implantation site of an electrode lead equipped with an electrode element, which are caused by the movement of the body or the beating of the heart, or by varying hard blood pressures. In such bending, the loads to the transition of the flexible feed line to the rigid electrode is of particular disadvantage, as high forces are created at the transitional site in the case of bending loads. The rigid, almost brittle joined transitional points create a problem with reliability in such electrode elements designed from the prior art, which is reflected in breakages and defective contacts at these points. Such defects are life-threatening to the patient, and are not repairable. This results in replacing the complete electrode lead, which also involves high risks.

Furthermore, the particular crimp connections between the feed line and electrode of an electrode element designed from the prior art comprise necessary additional connecting sleeves or pins for the safe connection as a counter bearing. This causes an enlargement of the diameter size, which is a disadvantage in that it disrupts the blood flow of intravascular, implantable electrodes, or the use of the same in a coronary blood vessel is no longer possible for the stimulation of the left half of the heart.

The present invention is directed towards overcoming one or more of the above-identified problems.

### SUMMARY OF THE INVENTION

The present invention is therefore based on the object of improving an electrode element of the above mentioned type with regard to the functionality thereof, and to avoid the risk of a defect due to breakage of the electrically conducting elements.

Said problem is solved by means of an electrically active electrode element for a medical electrode lead, including an electrode having an electrically active electrode surface facing toward the outside and an elongated electric feed line, which may establish an electric connection at the proximal end thereof to an electrically active medical device, and which is embodied as an electrically conducting cable end-to-end. Said electrode element is characterized in that the cable of the feed line forms the electrode at the distal end thereof.

An electric medical device may both be an external device, such as an external defibrillator, or any other device for stimulating a human or animal body, or for the diagnostic measuring of body signals. Particularly preferred are electrically active implants, such as pacemakers, cardioverters/defibrillators, or nerve stimulators. Other implants are also contemplated, which serve, for example, only to collect intracorporal signals and transmit the same to a receiver located outside of the body. Implants may also be utilized, which receive electric signals by means of electrode leads, but do not generate any electrotherapeutical stimulation, such as medicament dispensers.

An electrode element and the electrode lead of the present invention are characterized by an excellent break resistance, since one material cable forms both the feed line and the electrode end-to-end. Therefore, no joining or connection site is created that presents a tendency for breakages under alternating loads, and also contains no transition from a flexible to a rigid section.

Furthermore, the electrode element in accordance with the present invention is characterized in that a completely flexible electrode for small diameters and small stimulation surfaces may be provided for the first time.

It has further shown to be an advantage of the present invention that electrode elements, wherein the elongated feed line and the electrode are made from one material cable, have a high degree of electrical strength, by means of which both flexible use and stimulation electrodes in case of defibrillation electrodes may be provided.

According to an alternative embodiment, the distal end of the feed line cable is embodied in a tubular or in a cylindrical manner, thus forming the electrode having the electrically active electrode surface facing toward the outside, wherein preferably the longitudinal axis of the tube-shaped or cylindrically formed electrode is positioned as an extension of the elongated feed line.

Preferably, the tube-shaped electrode is embodied in a helical manner, in that the cable is wound or reeled about the longitudinal axis.

According to a further alternative embodiment of the electrode element, the distal end of the feed line cable is shaped in a meandering manner, and the electrode is configured as a planar surface having two exterior edges that are positioned at a right angle toward each other, whereby the electrically active electrode surface facing toward the outside is formed. For this purpose, the electrode is preferably positioned as an extension of the feed line, wherein particularly preferably the planar electrode is formed in a tubular or in a cylindrical manner.

Both alternatives attest to the flexible applicability of such electrode elements. The same may be utilized as implantable electrode lead intracardially, epicardially, in the coronary sinus, or for the stimulation of nerves. The same have a round cross-section across their full length. The electrode elements may also be utilized in patch electrode leads, which are a particular embodiment of epicardial electrode leads, and which allow for a large-scale stimulation outside of the heart. Other external applications are also conceivable, such as external defibrillation electrode leads, or even electrode leads by means of which non-invasive body signals can be measured.

Preferably, the cable forms a plurality of helical or adjacently positioned threads in the area of the electrode in both alternatives, which preferably are welded to each other, at least in sections.

This arrangement reduces the electrical resistance, thus further leading to a further increase of the electrical strength and reliability.

The cable of the electrode element is further preferably formed from drawn filled tube (“DFT”) wires and/or wires made from solid matter, wherein the core wires of each individual DFT wire or of the individual wire made from solid matter consists of one of the materials selected from molybdenum, tantalum, niobium, zirconium, or a platinum iridium alloy.

A further object of the present invention is an implantable electrode lead including an electrode lead boy having a proximal and a distal end, a plug positioned on the proximal end of the electrode lead body for the firm electric contacting of an electrically active implant, and at least one above mentioned electrode element.

Said object is based on the task of creating a flexible electrode lead that is as thin as possible, which is reliable, break-resistant, and simple to produce.

By using the electrode element the electrode lead obtains the same positive properties, such as a high break resistance, low diameter of the electrode lead body, and high electrical strength.

Preferably, the proximal end of the lead of the electrode element is connected to the plug at the proximal end of the electrode lead body in an electrically conducting manner, wherein the electrode is positioned at the distal end and/or in the distal end area of the electrode lead body.

The distal end area is the area located at the site in or on the body, body medium, and/or body tissue, at which the signals are to be received, and/or at which the body is to be stimulated.

In a further preferred embodiment, the electrode lead body has at least one recess at the distal end or in the distal area, preferably an annular groove or a lateral groove, in which the electrode is positioned in a dimensionally and positionally stable manner, wherein the electrically active electrode surface facing toward the outside is fitted into the electrode lead body such that the same is isodiametric or isoplanar with the surface insulated toward the outside.

Due to this arrangement, it is possible to produce an electrode lead which has no protrusions or projecting parts being bothersome during implantation or within the body. Safe and precise positioning and safe operation are therefore ensured.

In a further preferred manner, the feed line of the electrode element extends within the electrode lead body in an insulated manner and is guided outside of the electrode lead body in the area of the distal end thereof such that the electrode. Preferably, the electrically active electrode surface facing toward the outside has non-insulated contact with the body medium and/or body tissue.

Furthermore, the electrode lead body may be flexible and bendable in all embodiments of the electrode lead, and may preferably be made from a plastic, such as silicone or polyurethane. The electrode lead body of the electrode lead further includes a first bore for accommodating the feed line, and a second bore for temporarily accommodating a guide wire, wherein the first bore has a proximal end on or in the proximal end of the electrode lead body, and a distal end in or on a recess associated with the same.

Due to this innovative arrangement, the production of the electrode lead is facilitated. The production including few steps enables a highly automated and economical production of all implantable electrode leads. Furthermore, an adjusted and flexible production of various different implantable electrodes is facilitated.

As a further object the present invention is based on creating an economical, flexible, and easy method for the production of an electrode element and an electrode lead.

Said problem is solved in that the method for the production of the electrode element and the implantable electrode lead comprises the following steps:
producing the electrode element by means of forming the electrode from the cable of the feed line,
forming of the electrode lead body, and
attaching the plug and the electrode element to the electrode lead body.

Preferably, the forming step of the production of the electrode element includes the winding or reeling or meander-shaped forming of the distal end of the cable.

The step for forming the electrode lead body further preferably includes an injection molding method, wherein the recess and the first and second bore are formed.

Particularly preferred, the mounting step further includes the following steps:
threading the proximal end of the feed line into the first bore in or on the associated recess, and pushing the feed line in the direction toward the proximal end of the electrode lead body until the electrode comes to rest in the associated recess,
adhering the electrode to the electrode lead body in the recess, and
electrically connecting the proximal end of the feed line to the plug, and attaching the plug to the electrode lead body.

Further embodiment possibilities are explained in the description or in the drawings. Other objects, aspects and advantages of the present invention can be obtained from a study of the specification, the drawings, and the appended claims.

### BRIEF DESCRIPTION OF THE DRAWINGS

The invention is now described based on the figures. They show:

FIG. 1A shows an embodiment of an electrode element according to an embodiment of the present invention;

FIG. 1B is a cross-sectional illustration across the electrode of the electrode element from FIG. 1A along the cross-sectional line 1b;

FIG. 2A shows a further embodiment of the electrode element of the present invention:

FIG. 2B is a cross-sectional illustration across the electrode element from FIG. 2A along the cross-sectional line 2b

FIG. 3 shows an implantable electrode lead having an electrode element according to an embodiment of the present invention;

FIG. 4A shows an electrode lead body for an electrode lead having an electrode element according to an embodiment of the present invention;

FIG. 4B is a cross-sectional illustration across the recess of the electrode lead body along the cross-sectional line 4b in FIG. 4A:

FIG. 4C is a cross-sectional illustration across the electrode lead body along the cross-sectional line 4c in FIG. 4A;

FIG. 5A shows an electrode lead body for a multipolar implantable electrode lead according to an embodiment of the present invention;

FIG. 5B is a cross-sectional illustration of the electrode lead body for multipolar implantable electrode leads along the cross-sectional lines 5b in FIG. 5A;

FIG. 5C is a cross-sectional illustration of the electrode lead body for multipolar implantable electrode leads along the cross-sectional lines 5c in FIG. 5A; and

FIG. 5D is a cross-sectional illustration of the electrode lead body for multipolar implantable electrode leads along the cross-sectional lines 5d in FIG. 5A;

### DETAILED DESCRIPTION OF THE INVENTION

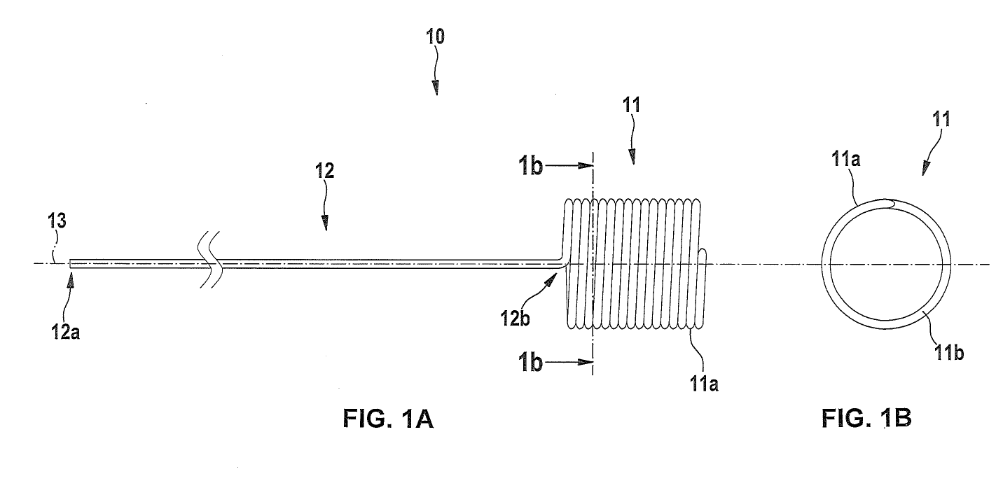

FIGS. 1A and 1B show an electrode element 10 according to a first embodiment, comprising an electrode 11 and a feed line 12, and a cross-section across the electrode 11. For this purpose, the feed line 12 consists of a cable made from solid wire or casing wire strands or fibers of the type cited above. The distal end of the cable forms the electrode 11, in that in this embodiment the wire is wound or reeled in a helical manner along a centrally positioned longitudinal axis 13. The feed line 12 is positioned as an extension of said longitudinal axis 13 and may be positioned either parallel to the same or on said longitudinal axis. The electrode 11 is therefore positioned directly distal of the distal end 12b of the feed line 12.

The electrode 11 further forms an electrically active electrode surface 11a facing toward the outside (opposite of the longitudinal axis 13), which is in contact with the body medium and/or body tissue in the mounted state. Due to the helically wound manner the cable forms threads 11b. For this purpose, a thread is a revolution of the wire about the longitudinal axis 13. A further thread is formed in each additional revolution. The threads 11b are preferably positioned directly adjacent to subsequent distal or preceding proximal threads, but may also be embodied in a different manner, for example, in that an intermediate space filled with insulation material is present between each thread 11b. In this manner, the electrode surface 11a is generally interrupted by an intermediate space between each thread 1b.

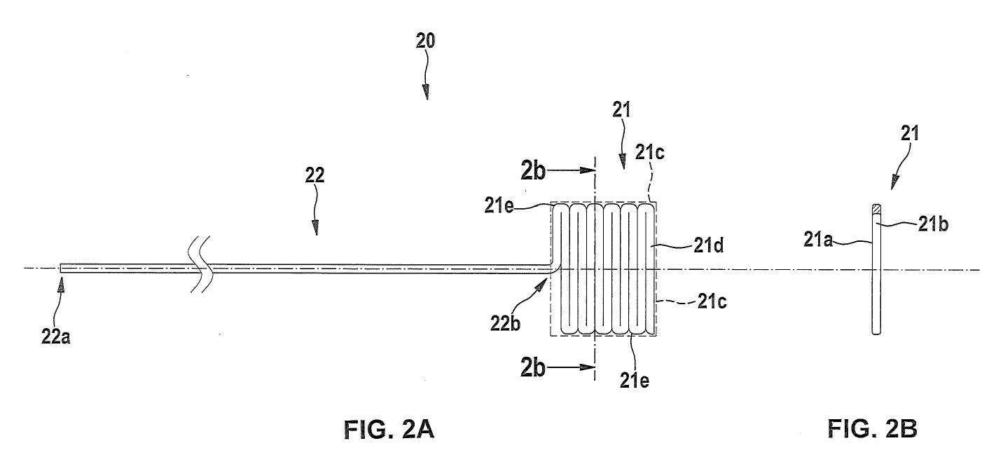

A further embodiment of the electrode element 20 is shown in FIGS. 2A and 2B. There, the electrode 21 is not formed by means of helical winding or reeling of the cable, but by means of a meandering zigzag formation consisting of straight sections 21d, the opposite ends thereof are connected both to the distal preceding and to the proximal subsequent straight section 21d by means of opposite bent sections 21e. Therefore, contrary to previous embodiments, an electrode surface 21a that is planar and facing toward the outside is formed, having two intended outer edges 21c that are positioned at a right angle to each other.

This arrangement also forms threads 21b extending through a straight section from one bent section at one end to the bent section at the other end of the straight section.

The electrode 21 is positioned as an extension to the elongated feed line 22 at the distal end 22b thereof, and is also formed by the distal end of the feed line cable.

In said configuration, this electrode element may be utilized, for example, in a so-called patch electrode lead, or even in an external recognition electrode lead that can be placed onto the body. Preferably, however, a planar, flat electrode 21 is formed in a tubular or in a cylindrical manner along a longitudinal axis, wherein the elongated feed line 22 is positioned on or parallel to said longitudinal axis. This creates a configuration that is simple to produce, which may be utilized, for example, in an implantable electrode lead.

In both embodiments, individual, multiple, or all threads 11b and 21b may be welded to each other, for example, by means of laser or resistance welding or the like. This may be carried out, for example, by means of alternating welding of threads 11b in pairs at locations that are opposite of each other in circumferential direction in order not to reduce flexibility.

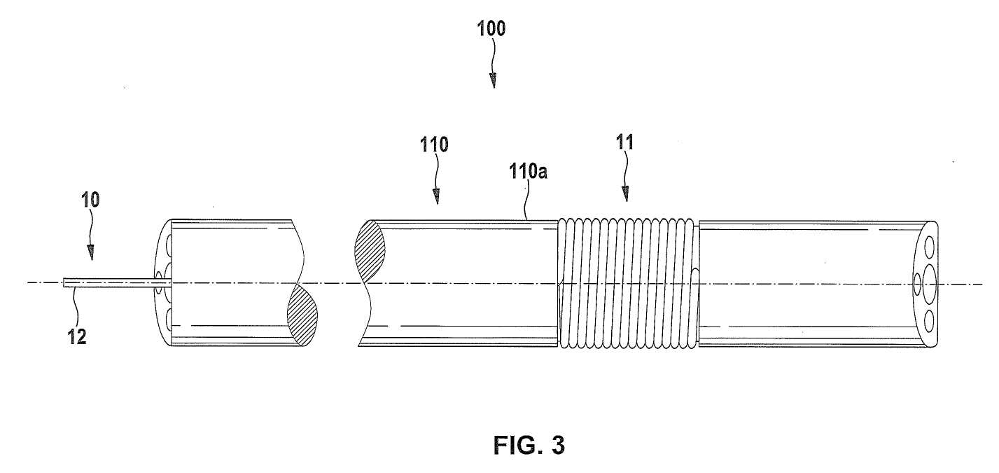

FIG. 3 shows the section of an implantable electrode lead 100 comprising an electrode element 10. The electrode element 10 is incorporated only into one electrode lead body 110 such that the electrode 11 is positioned isodiametrically toward the outer insulated surface 110a of the electrode lead body. The feed line 12 extends along the longitudinal axis of the electrode lead body 110 in order to be electrically connected to a plug located and mounted on the distal end of the electrode lead body. Said plug should correspond with the IS-1, DF-1, and IS-4 standards, or standards yet to be developed, by means of which an electric connection may be established to an electrically active implant of the type mentioned above.

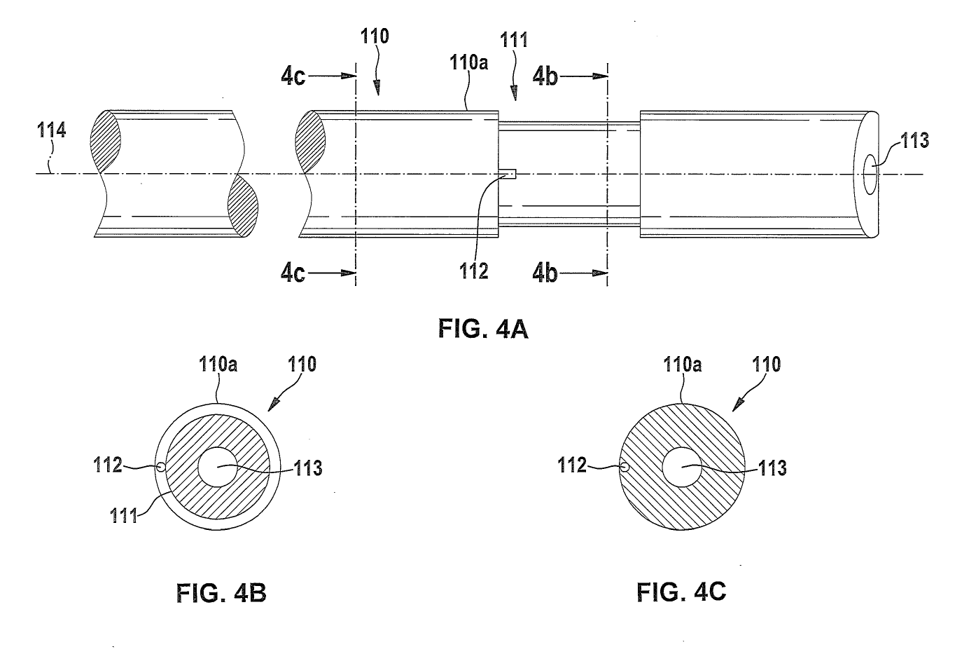

The electrode lead body 110 is illustrated in detail in FIGS. 4A to 4C. The electrode lead body has a recess 111 in the form of an annular groove on or near the end thereof facing away from the plug, which includes a radial depth corresponding to the thickness of the cable, and thus of the electrode 11. This means that the diameter of the electrically active surface 11a facing toward the outside corresponds to the largest diameter of the electrode lead body 110 at the point of the largest diameter thereof on the outer insulated surface, while the inner diameter of the electrode 11 corresponds to the outer diameter of the recess 111.

A first bore 112 is in contact with the recess 111 at the distal end thereof, wherein said bore extends parallel to the longitudinal axis 114 up to the proximal end of the electrode lead body 110. In the assembled state of the implantable electrode lead 100, said bore 112 accommodates the feed line 12, which is guided outside of the bore of the electrode lead body 110 at the distal end 12b thereof and into the recess 111. Both the electrode 11 and the feed line 12 may be adhered in the recess 111 or in the bore 112.

Furthermore, such an electrode lead body 110 may further have a second bore 113, by means of which the implantable electrode lead 100 may be guided using a guide wire. The same is usually positioned on the longitudinal axis 114. Additionally, the electrode may comprise the common active and passive mountings known from prior art.

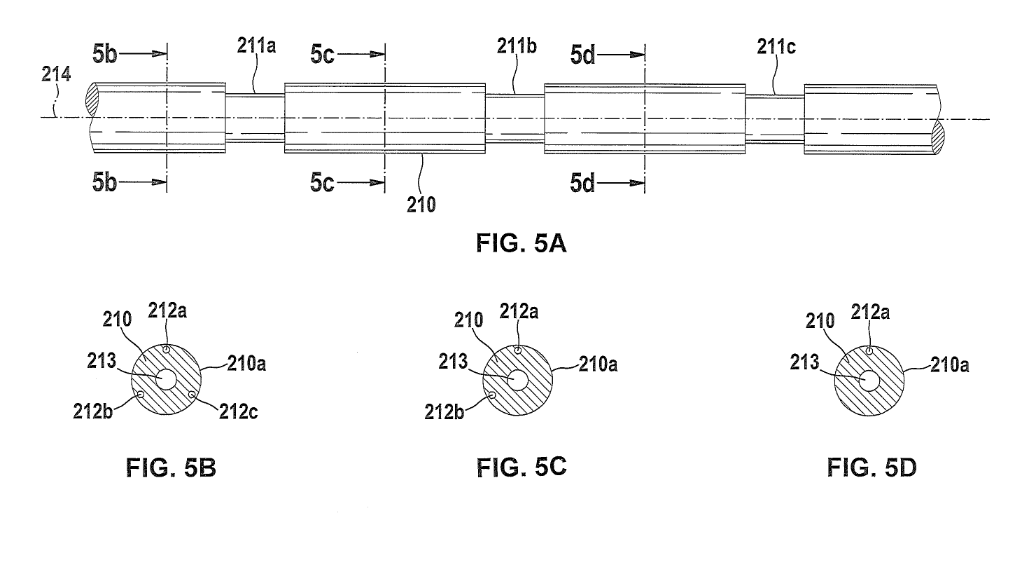

In a further embodiment of the implantable electrode lead, the same may be embodied in a multipolar manner, i.e., having two or more electrodes. For this purpose, in, for example, a three-polar electrode lead, the electrode lead body 210 may comprise, for example, the construction shown in FIGS. 5A to 5D. The same then includes three combinations of recesses 211a, 211b, and 211c similar to those described in FIGS. 4A to 4C, wherein bores 212a, 212b, and 212c are associated with the same in connection with one of said recesses. Each of these bores extends from a distal end positioned in or on the associated recesses along the longitudinal axis 214 of the electrode lead body 210 up to the proximal end of the electrode lead body, at which a known plug is mounted for the electrical connection to an implant. As the recesses 211a, 211b, and 211c, all of which are located in the distal area near the distal end, are positioned in various different positions along the longitudinal axis of the electrode lead body 210, the bores 212a, 212b, and 212c are of varying lengths. In order to ensure the necessary insulation against the other feed lines 12 the bores are advantageously distributed evenly, or equi-angularly, in a circumferential direction about the longitudinal axis 214, or about the bore 213. Other configurations for the bores are also contemplated. With this construction, even more implantable electrode leads comprising even more electrodes are possible.

It will be apparent to those skilled in the art that numerous modifications and variations of the described examples and embodiments are possible in light of the above teachings. The disclosed examples and embodiments are presented for purposes of illustration only and are not meant to limit the scope of the invention in any way. Therefore, it is the intent to cover all such modifications and alternate embodiments as may come within the true scope of this invention, which is to be given the full breadth of the appended claims and any and all equivalents thereof.

We claim:

1. An electrically active electrode element for a medical electrode lead, comprising:
an electrode having an electrically active electrode surface facing toward the outside; and
an elongated electric feed line having proximal and distal ends, which is capable of establishing an electric connection to an electrically active medical device at the proximal end, and which is embodied as an electrically conducting cable end-to-end, wherein the cable forms the electrode at the distal end.

2. The electrode element according to claim 1, wherein the distal end of the feed line cable is formed as a tube or in a cylindrical manner forming the electrode having the electrically active electrode surface facing toward the outside, wherein a longitudinal axis of the electrode formed as a tube or in a cylindrical manner is positioned as an extension to the elongated feed line.

3. The electrode element according to claim 2, wherein the electrode is formed as a tube in a helical manner, wherein the cable is wound about the longitudinal axis.

4. The electrode element according to claim 1, wherein the distal end of the feed line cable is formed in a meandering manner, and that the electrode is therefore embodied as a planar surface having two intended outer edges that are positioned at a right angle toward each other and forms the electrically active electrode surface facing toward the outside.

5. The electrode element according to claim 4, wherein the flat electrode is formed in a tubular or in a cylindrical manner.

6. The electrode element according to claim 3, wherein the cable forms a plurality of helical or adjacently positioned threads in the area of the electrode.

7. The electrode element according to claim 6, wherein the helical or adjacently positioned threads are welded to each other at least in sections.

8. The electrode element according to claim 1, wherein the cable is formed of drawn filled tube (DFT) wires and/or wires made from solid matter.

9. The electrode element according to claim 8, wherein the core wire of each individual DFT wire, or of the individual wire made from solid matter, is made from one of the materials selected from molybdenum, tantalum, niobium, zirconium, or a platinum iridium alloy.

10. An implantable electrode lead, comprising:
an electrode lead body having a proximal and a distal end;
a plug positioned at the proximal end of the electrode lead body for the firm electrical contacting of an electrically active implant; and
at least one electrode element according to claim 1.

11. The electrode lead according to claim 10, wherein the proximal end of the feed line of the electrode element is connected to the plug in an electrically conducting manner at the proximal end of the electrode lead body, and that the electrode is positioned at the distal end and/or in the distal end area of the electrode lead body.

12. The electrode lead according to claim 10, wherein the feed line of the electrode element extends within the electrode lead body in an insulated manner, and is guided outside of the electrode lead body in the area of the distal end thereof such that the electrically active electrode surface facing toward the outside has non-insulated contact with the body medium and/or body tissue.

13. The electrode lead according to claim 10, wherein the electrode lead body has at least one recess at the distal end, or in the distal area, the at least one recess including an annular groove or a lateral groove, in which the electrode is positioned in a dimensionally and positionally stable manner, wherein the electrically active electrode surface facing toward the outside is fitted into the electrode lead body such that it is isodiametric or isoplanar with the outer insulated surface.

14. The electrode lead according to claim 10, wherein the electrode lead body is flexible and bendable, and is made of a plastic, and has at least one first bore for accommodating the feed line, and a second bore for temporarily accommodating a guide wire, wherein the at least one first bore has a proximal end on or in the proximal end of the electrode lead body, and a distal end in or on a recess associated with the same.

15. The electrode lead according to claim 14, wherein the plastic comprises silicone or polyurethane.

16. A method for the production of an electrode element and an implantable electrode lead according to claim 1, comprising the following steps:
producing the electrode element by means of forming the electrode from the cable of the feed line;
forming of the electrode lead body; and
attaching the plug and the electrode element on the electrode lead body.

17. The method according to claim 16, wherein the forming step of the production of the electrode element comprises the winding or reeling of the distal end of the cable.

18. The method according to claim 16, wherein the forming step of the production of the electrode element comprises the meandering forming of the distal end of the cable

19. The method according to claim 12, wherein the step of forming the electrode lead body comprises an injection molding method, wherein recesses and the first and second bores are integrally molded.

20. The method according to claim 19, wherein the attaching step comprises the following steps:
threading the proximal end of the feed line into the first bore in or on the associated recess, and pushing the feed line in the direction toward the proximal end of the electrode body until the electrode comes to rest in the recess;
adhering the electrode to the electrode lead body in the recess; and
electrically connecting the proximal end of the feed line to the plug, and attaching the plug to the electrode lead body.
